(19) United States
(12) Patent Application Publication
(10) Pub. No.: US 2026/0118361 A1
(43) Pub. Date: Apr. 30, 2026

(54) FLUOROGENIC BIOCONJUGATION OF CYCLOPROPANOL-BASED FLUOROPHORES FOR BIOLOGICAL APPLICATIONS

(71) Applicant: UNIVERSITY OF SOUTH CAROLINA, COLUMBIA, SC (US)

(72) Inventors: Qian Wang, COLUMBIA, SC (US); Shan Hu, COLUMBIA, SC (US)

(21) Appl. No.: 19/365,638

(22) Filed: Oct. 22, 2025

Related U.S. Application Data
(60) Provisional application No. 63/711,348, filed on Oct. 24, 2024.
(60) Provisional application No. 63/813,851, filed on May 29, 2025.

(51) Int. Cl. G01N 33/58; C07C 43/23; C07D 311/16

(52) U.S. Cl. CPC G01N 33/582; C07C 43/23; C07D 311/16

(57) ABSTRACT

In general, disclosed herein are fluorogenic compounds having the structure of Formula (I):

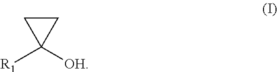

Fluorogenic compounds disclosed herein may be useful in methods for visualizing a sample. The method may include conjugating the biomolecule with sample comprising a fluorogenic compound disclosed herein; incubating the biomolecule with the fluorogenic compound for a sufficient time to allow for fluorogenic bioconjugation of the fluorogenic compound; subjecting the fluorogenic compound to an oxidative condition; contacting the fluorogenic compound with a dye; and imaging the fluorogenic compound, thereby determining the fluorescence intensity change of the fluorogenic compound.

### CROSS REFERENCE TO RELATED APPLICATIONS

This application claims the filing benefit of U.S. Provisional Patent Application Ser. No. 63/711,348, filed on Oct. 24, 2024, and U.S. Provisional Patent Application Ser. No. 63/813,851, filed on May 29, 2025, which are incorporated herein by reference.

### BACKGROUND

The fluorescent labeling of proteins is crucial for studying localization, trafficking, and interaction networks in live cells. However, the challenges of removal of high background signals caused by excess dye or incomplete removal of unreacted fluorophores limit the use of traditional protein labeling in live cells. Fluorogenic bioconjugation overcomes this limitation by using minimally fluorescent precursors that become highly emissive upon covalent attachment to target biomolecules. This turn-on behavior-driven by intramolecular charge transfer (ICT) in the newly formed conjugate-yields a low background and enables real-time visualization in diverse biological contexts.

Several strategies for fluorogenic labeling of biomolecules have been previously reported. Common methods include the design of fluorophores that are initially non-emissive or weakly fluorescent and become highly emissive upon specific chemical reactions, such as enzymatic cleavage, bond formation, or environmental changes. For example, azide-alkyne cycloaddition or click chemistry has been widely used with fluorogenic probes that become fluorescent after the formation of a triazole ring. Another approach such as tetrazine ligation with strained alkenes. Despite these advances, existing fluorogenic systems often lack spatial or temporal control for dynamic labelling in the biological context.

Despite the remarkable achievements of these three-member ring systems in bioconjugation, the options for utilizing the cyclopropene warhead in bioconjugation remain limited, the diazirine tag exhibits reduced chemical specificity due to its heightened reactivity upon exposure to light, and the size of oxaziridine substitution increased the potential for interference with live cells. Collectively, these limitations underscore the need to develop an improved bioconjugation reporter to enhance its applicability and selectivity in biological applications

### SUMMARY

In general, disclosed herein are fluorogenic compounds. The fluorogenic compounds may have the structure of Formula (I):

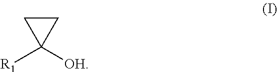

Additionally, disclosed herein are methods for visualizing a biomolecule in diverse biological systems. The method may include conjugating the biomolecule with sample comprising a fluorogenic compound disclosed herein; incubating the biomolecule with the fluorogenic compound for a sufficient time to allow for fluorogenic bioconjugation of the fluorogenic compound; subjecting the fluorogenic compound to an oxidative condition; contacting the fluorogenic compound with a dye; and imaging the fluorogenic compound, thereby determining the fluorescence intensity change of the fluorogenic compound.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A depicts prediction of the occurrence of intramolecular charge transfer (ICT). The electron distribution and HOMO-LUMO energy gap of Npt-CPol I-1 were calculated both before and after ring-opening reactions. All calculations were performed using density functional theory calculation at the B3LYP-D3/6-311+G* level of theory with Spartan'18.

FIG. 1B depicts prediction of the occurrence of intramolecular charge transfer (ICT). The electron distribution and HOMO-LUMO energy gap of Coumarin-CPol I-2 were calculated both before and after ring-opening reaction. All calculations were performed using density functional theory calculation at the B3LYP-D3/6-311+G* level of theory with Spartan'18.

FIG. 2A depicts absorption and fluorescence spectra of fluorogenic dyes Npt-CPol I-1 (100 μM in H₂O) and their iodo-substituted ketones I-1a (100 μM in H₂O). The excitation wavelengths were set at 330 nm for I-1 and I-1a.

FIG. 2B depicts absorption and fluorescence spectra of fluorogenic dyes Coumarin-CPol I-2 (10 μM in H₂O) and their iodo-substituted ketones coumarin-iodo-ketone I-2a (10 μM in H₂O). The excitation wavelengths were set at 423 nm for I-2 and I-2a.

FIG. 3A illustrates bioconjugation between intermediate I-1a and peptide bivalirudin in pH 7.0 KP buffer for 3 hours at room temperature.

FIG. 3B depicts MALDI-TOF MS analysis of before and after conjugation of bivalirudin (M=2179 Da) with I-1a.

FIG. 4A illustrates the electrochemical bioconjugation reaction between CPol-containing naphthalene fluorogenic dyes (I-1) and biomolecules.

FIG. 4B depicts fluorescence spectrometry of the angiotensin I starting material mixture and in situ-generated conjugates were performed at identical concentrations, excitation settings (317 nm), and voltage conditions (800 V) as those used for I-1a and I-1b.

FIG. 4C depicts fluorescence spectrometry of the lysozyme starting material mixture and in situ-generated conjugates were performed at identical concentrations, excitation settings (317 nm), and voltage conditions (800 V) as those used for I-1a and I-1b.

FIG. 4D depicts MALDI-TOF MS analysis of conjugation of angiotensin I with Coum-CPol I-2 with NaI (IIa) at 0.8 V and with NaBr (IIb) at a constant voltage of 1.2 V.

FIG. 4E depicts MALDI-TOF MS analysis of conjugation of lysozyme with Coum-CPol I-2 with NaI (IIa) at 0.8 V and with NaBr (IIb) at a constant voltage of 1.2 V.

FIG. 5A illustrates the electrochemical bioconjugation reaction between CPol-containing coumarin fluorogenic dyes (I-2) and biomolecules.

FIG. 5B depicts fluorescence spectrometry of the starting material mixture and in situ-generated conjugates were performed at identical concentrations, excitation settings (423 nm), and voltage conditions (800 V) as those used for I-2a and I-2b.

FIG. 5C depicts MALDI-TOF MS analysis of conjugation of lysozyme with Coum-CPol I-2 with NaI (IIa) at 0.8 V and with NaBr (IIb) at a constant voltage of 1.2 V.

FIG. 6A illustrates SDS-PAGE analysis of BSA conjugated with fluorogenic dyes I-1 and I-2 under optimized electrochemical conditions or with β-iodo-substituted ketones (I-1a and I-2a) under the same conditions. CBB: Coomassie brilliant blue staining; WB-FL: Western blot fluorescence imaging; FL: In-gel fluorescence imaging.

FIG. 6B illustrates SDS-PAGE analysis of bGus conjugated with fluorogenic dyes I-1 and I-2 under optimized electrochemical conditions or with β-iodo-substituted ketones (I-1a and I-2a) under the same conditions. CBB: Coomassie brilliant blue staining; WB-FL: Western blot fluorescence imaging; FL: In-gel fluorescence imaging.

Repeat use of reference characters in the present specification and drawings is intended to represent the same or analogous features or elements of the present invention.

### DETAILED DESCRIPTION

### Definitions

It is to be understood that the terminology used herein is for the purpose of describing particular embodiments only and is not intended to limit the scope of the present disclosure. In this specification and in the claims that follow, reference will be made to a number of terms that shall be defined to have the following meanings.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components, or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximate unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

As used herein, an “alkyl” group may refer to a straight or branched chain hydrocarbon, having a certain number of carbon atoms (e.g., C₁₋₁₂ carbon atoms). For instance, an alkyl group may include, but is not limited to, straight and branched chain alkyl groups having 1, 2, 3, 4, 5, 6, 7, 8, 9, 10, 11 or 12 carbon atoms. Alternatively, an alkyl group may include, but is not limited to, methyl, ethyl, n-propyl, isopropyl, n-butyl, t-butyl, isobutyl, sec-butyl, n-pentyl, n-hexyl, n-heptyl, n-octyl, or a combination thereof.

As used herein, an “aryl” group may refer to, by itself or as part of another substituent, may include, but is not limited to, a monocyclic, bicyclic or polycyclic polyunsaturated aromatic hydrocarbon radical containing 6 to 14 ring carbon atoms, which may be a single ring or multiple rings (up to three rings) which are fused together or linked covalently.

As used herein, an “alkenyl” group may refer to a linear monovalent hydrocarbon radical or a branched monovalent hydrocarbon radical having the number of carbon atoms indicated in the prefix and containing at least one double bond. Additionally, as used herein, “alkynyl” may refer to a linear monovalent hydrocarbon radical or a branched monovalent hydrocarbon radical containing at least one triple bond and having the number of carbon atoms indicated in the prefix.

As used herein, an “alkoxy” group may refer to an —O-alkyl group, where alkyl is as defined herein. Similarly, an “halogen substituted alkoxy” may refer to an alkoxy in which the alkyl group is substituted with one or more halogen atoms. Additionally, “thioalkoxy” group may refer to an —O-alkylthio group, where the alkylthio group may include, but is not limited to, thiomethoxy, thioethoxy, and the like.

As used herein, a “halogen” group may refer to an element found in Group 17 (formerly Group VIIA) of the periodic table. For instance, a halogen may include, but is not limited to, fluorine (F), chlorine (Cl), bromine (Br), iodine (I), astatine (At), and the like.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure covers such modifications and variations as come within the scope of the appended claims and their equivalents.

Disclosed herein are fluorogenic compounds that may act as a useful fluorescent probe in various biological applications. For instance, fluorogenic compounds disclosed herein may function as a bioconjugation warhead in various biological applications. As used herein, “bioconjugation warhead” refers to a reactive functional group or moiety that may be used in chemical reactions to attach biomolecules to other molecules. For instance, the bioconjugation warhead may target a specific site on a biomolecule.

In some example embodiments, the fluorogenic compound may include, but is not limited to, a saturated or unsaturated, aromatic or non-aromatic, monocyclic, bicyclic or tricyclic carbon ring system. For instance, in some example embodiments, the fluorogenic compound may include, but is not limited to, a non-aromatic monocyclic carbon ring system. In another example embodiment, the fluorogenic compound may be a non-aromatic bicyclic carbon ring system.

In some example embodiments, the fluorogenic compound may be a three-member ring system. For instance, in some example embodiments, the three-member ring may include, but is not limited to, cyclopropanol (CPol) or a substituted CPol. The substituted CPol may further include, but is not limited to, hydroxyl, amino, alkyl, aryl, alkenyl, alkynyl, alkoxy, carboxy, benzyl, phenyl, nitro, thiol, thioalkoxy, halogen, or a combination thereof. In one example embodiment, the substituted CPol may include hydroxyl group. In another example embodiment, the substituted CPol may include an amino group. In another example embodiment, the substituted CPol may include an alkyl group. In another example embodiment, the substituted CPol may include an aryl group. In another example embodiment, the substituted CPol may include an alkenyl group. In another example embodiment, the substituted CPol may include an alkynyl group. In another example embodiment, the substituted CPol may include an alkoxy group. In another example embodiment, the substituted CPol may include a carboxy group. In another example embodiment, the substituted CPol may include a benzyl group. In another example embodiment, the substituted CPol may include a phenyl group. In another example embodiment, the substituted CPol may include a nitro group. In another example embodiment, the substituted CPol may include a thiol group. In another example embodiment, the substituted CPol may include a thioalkoxy group. In another example embodiment, the substituted CPol may include a halogen.

Advantageously, cyclopropanol (CPol)-based fluorogenic compounds disclosed herein may be useful in spatiotemporally controlled visualizing dynamic behaviors in the biological context. Upon activation by oxidative or electrochemical stimuli, the CPol scaffolds undergo ring-opening to form electrophilic β-halide-substituted ketones, enabling selective conjugation with nucleophilic biomolecules such as peptides and proteins. This platform allows for real-time, low-background fluorescent labeling in vitro and in live-cell environments.

In some example embodiments, the CPol derivative may have the following Formula (I),

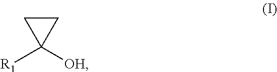

wherein R₁ may be selected from a group consisting of

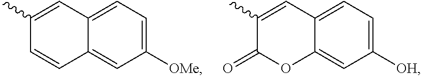

hydroxyl, amino, alkoxy, carboxy, benzyl, phenyl, nitro, thiol, thioalkoxy, halogen, alkyl, aryl, alkenyl, alkynyl, halogen, C₁₋₆alkyl, phenyl, perdeuterated phenyl, benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl, 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbamoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, or a combination thereof. In one example embodiment, R₁ may include, but is not limited to, phenyl, toyl, benzyl, phenyl, methoxyphenyl, methyl, fluoromethyl, trifluoromethyl, propyl, isopropyl, or a combination thereof. In another example embodiment, R₁ may be

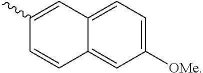

In yet another example embodiment, R₁ may be

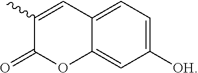

In one example embodiment, the CPol derivative may include, but is not limited to, 1-(6-methoxynaphthalen-2-yl)cyclopropan-1-ol (Npt-CPol), 7-hydroxy-3-(1-hydroxycyclopropyl)-2H-chromen-2-one (Coumarin-CPol), phenylcyclopropanol, 1-(p-tolyl)cyclopropan-1-ol, benzylcylopropanol, pentylcyclopropanol, 1-(4-methoxyphenyl)cyclopropan-1-ol, 1-(4-(trifluoromethyl)phenyl)cyclopropan-1-ol, 1-(3-methoxyphenyl)cyclopropan-1-ol, 1-(3-(trifluoromethyl)phenyl)cyclopropan-1-ol, 1-isopropylcyclopropan-1-ol, 3-bromopropyl cyclopropanol, or a combination thereof.

In one example embodiment, the substituted CPol may be 1-(6-methoxynaphthalen-2-yl)cyclopropan-1-ol (Npt-CPol). For instance, 1-(6-methoxynaphthalen-2-yl)cyclopropan-1-ol (Npt-CPol) may have the following Formula (I-1):

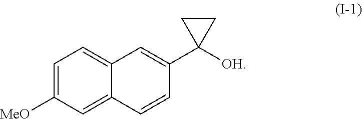 [SMILES: COc1ccc2cc(C3(O)CC3)ccc2c1]

In one example embodiment, the substituted CPol may be 7-hydroxy-3-(1-hydroxycyclopropyl)-2H-chromen-2-one (Coumarin-CPol). For instance, 7-hydroxy-3-(1-hydroxycyclopropyl)-2H-chromen-2-one (Coumarin-CPol) may have the following Formula (I-2):

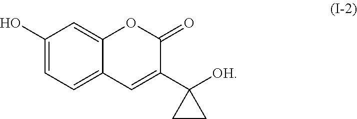 [SMILES: O=c1oc2cc(O)ccc2cc1C1(O)CC1]

In some example embodiments, the fluorogenic compound may include, but is not limited to, a halogen donor compound. As used herein, a “halogen donor” compound refers to a compound that can release or donate a halogen atom in a chemical reaction. For instance, a halogen donor compound may donate said halogen atom during an electrochemical process to form a compound which includes said halogen atom.

In some example embodiments, the halogen donor compound may include, but is not limited to, a metal. salt metal may include an alkali metal. For instance, the metal may include, but is not limited to, sodium, lithium, potassium, rubidium, aluminum, cesium, or francium. In one example embodiment, the metal may be sodium. In another example embodiment, the metal may be potassium. In another example embodiment, the metal may be lithium. In another example embodiment, the metal may be aluminum.

In some example embodiments, the halogen donor compound may be a sodium based compound. For instance, in some example embodiments, the sodium based compound may have the following Formula (II):

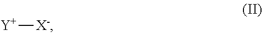

wherein Y may be a metal; and X may be a halogen. In some example embodiments, the halogen donor compound may include, but is not limited to, sodium iodide, sodium bromide, lithium bromide, lithium iodide, potassium bromide, potassium iodine, tetraethylammonium bromide, tetraethylammonium chloride, or a combination thereof.

In one example embodiment, the halogen donor compound may comprise sodium iodide. For instance, sodium iodide may have the following Formula (IIa):

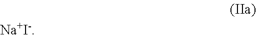

In one example embodiment, the halogen donor compound may comprise sodium bromide. For instance, sodium bromide may have the following Formula (IIb):

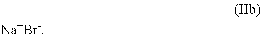

In general, disclosed herein are methods for producing a chemoselectively modified biomolecule. The method may include providing a biomolecule, which may include a bioconjugation warhead at a specific site of the biomolecule. Also, the method may include selectively oxidizing the halogen donor compound of the bioconjugation warhead at an electrochemically efficient voltage to generate a halogen radical. Also, the method may include contacting the halogen radical with the bioconjugation warhead at a constant voltage to form an electrophilic warhead. Also, the method may include reacting the electrophilic warhead with a coupling partner comprising a nucleophilic group of the biomolecule to conjugate the electrophilic warhead to the biomolecule.

In one example embodiment, the biomolecule may include, but is not limited to, nucleic acids, proteins, peptides, viruses, and the like. For instance, the biomolecule may be a nucleic acid. In another example embodiment, the biomolecule may be a protein. In another example embodiment, the biomolecule may be a peptide. In one example embodiment, the biomolecule may be bivalirudin. In another example embodiment, the biomolecule may be Angiotensin I. In another example embodiment, the biomolecule may be lysozyme. In another example embodiment, the biomolecule may be bovine serum albumin (BSA). In another example embodiment, the biomolecule may be β-glucuronidase (bGus). In another example embodiment, the biomolecule may be tobacco mosaic virus (TMV).

Also, the present disclosure is directed to a method of forming a bioconjugated molecule. For instance, an in situ electrophilic warhead may be formed in an electrochemical system. For instance, the electrochemical system may include one or more electrochemical cells with a reference electrode, a working electrode, and a cathode electrode. In one example embodiment, the reference electrode may be formed from any electrode material known in the art. For instance, the reference electrode may be formed from silver, silver chloride, or alloys, or a combination thereof. Nonetheless, the reference electrode may be selected from a material that is capable of delivering a current, such as a direct current (DC) or alternating current (AC) voltage signal. For instance, as generally known in the art, the reference electrode is selected and formed so as to form a stable voltage at the interface during the time of the measurement. An Ag/AgCl reference electrode is therefore often used for that reason.

In one example embodiment, the anode may include, but is not limited to, zinc, iron, chromium, nickel, lead, titanium, copper, tin, silver, lead (IV) oxide, manganese (IV) oxide, sulfur, Prussian blue, Prussian blue derivatives, transition metal analogs of Prussian blue, carbon fiber, graphite, carbon felt, conductive carbon black, as well as other conductive forms of carbon. For instance, the anode may include one or more layers including, without limitation, a separator, e.g., a porous carbon paper, carbon cloth, carbon felt, or metal cloth (e.g., a porous film made of fiber-type metal or a metal film formed on the surface of a polymer fiber cloth), a conductive substrate appropriate for the electrode electrolyte solution of the cell (e.g., graphite), and a current collector (e.g., gold-plated copper). In one example embodiment, the cathode may include, but is not limited to, nickel, iron, copper, ruthenium, platinum, palladium, rhodium, iridium, rhenium, silver, gold, or a combination thereof.

In one example embodiment, the electrochemical system may further include an electrolyte solution. For instance, the electrode electrolyte solution may be an aqueous solution that includes a redox component. The redox component may be oxidized at a first electrode and reduced at a second electrode as the electrode electrolyte solution is cycled through the electrochemical cell under a voltage potential established between the first and second electrodes.

In one example embodiment, the electrode electrolyte solution may include, but is not limited to, at least one redox component that is capable of a reversible redox reaction under the voltage potential of the electrochemical cell, providing a reversible redox pair. By way of example, and without limitation, the redox component can include one or more of, and without limitation to, an ion of titanium (titanium(III), titanium(IV)), vanadium (vanadium(II), vanadium(III), vanadium(IV), vanadium(V)), chromium (chromium(II), chromium(III), chromium(VI)), manganese (manganese(II), manganese(III), manganese(VI), manganese(VII)), iron (iron(II), iron(III), iron (VI)), cobalt (cobalt(II), cobalt(III)), nickel (nickel(II)), copper (copper(I), copper(II)), zinc (zinc(II)), ruthenium (ruthenium(II), ruthenium(III)), tin (tin(II), tin(IV)), cerium (cerium(III), cerium(IV)), tungsten (tungsten(IV), tungsten(V)), osmium (osmium(II), osmium(III)), lead (lead II), zincate, aluminate, chlorine, chloride, bromine, bromide, tribromide, iodine, iodide, triiodide, polyhalide, halide oxyanion, sulfide, polysulfide, sulfur oxyanion, ferrocyanide, ferricyanide, a quinone derivative, an alloxazine derivative, a flavin derivative, a viologen derivative, a metallocene derivative (e.g., a ferrocene derivative), a nitroxide radical derivative, a N,N-dialkyl-N-oxoammonium derivative, a nitronyl nitroxide radical derivative, and/or polymers incorporating complexed or covalently bound components of any of the aforementioned materials.

By way of example, a redox component can be present in the electrode electrolyte solution as a salt such as, and without limitation to, cerium chloride, germanium chloride, vanadium chloride, europium chloride, or ferrous chloride.

A redox pair of the electrode electrolyte solution can be an anion-based pair or a cation-based pair. For instance, a solution can include an anion-based redox component such as an aluminum-based Al(OH)₄⁻/Al redox pair, a zinc-based Zn(OH)₄²⁻/Zn redox pair, a sulfur-based S₄²⁻/S₂²⁻ redox pair, a cobalt-based Co(CN)₆³⁻/Co(CN)₆⁴⁻, or a bromine Br₃⁻/Br⁻ redox pair. Examples of cation-based redox components can include, without limitation, vanadium based redox pairs such as vanadium-based VO⁺/VO²⁺ or V³⁺/V²⁺ redox pairs, a zinc-based Zn²⁺/Zn redox pair, a cerium-based Ce⁴⁺/Ce³⁺ redox pair, a chromium-based Cr³⁺/Cr²⁺ redox pair, an iron-based Fe³⁺/Fe²⁺ redox pair, a cobalt-based Co³⁺/Co²⁺ redox pair, etc.

In addition to one or more compounds capable of providing the desired redox pair, the electrode electrolyte solution can include one or more solutes and solvents, pH buffers, etc. as are generally known in the art. For instance, a solution can include a pH buffer that may or may not be redox-active under typical operating conditions. In one example embodiment, the pH of the electrode electrolyte solution can be matched to the pH of the salt solution that includes the targeted ion. As such, the electrode electrolyte solution can be approximately neutral (e.g., pH from about 5 to about 9), acidic (e.g., pH less than about 5), or alkaline (e.g., pH greater than about 9), depending upon the characteristics of the system and the particular method.

In one example embodiment, the electrochemical system may further include one or more ion exchange membranes. For instance, the ion exchange membranes may be anion exchange membranes or cation exchange membranes, depending on the nature of the ion targeted for separation by the system and the redox component of the electrode electrolyte solution. While both ion exchange membranes will be either anion exchange membranes or cation exchange membranes, they can be of the same composition or different, as desired.

The ion exchange membranes may be water permeable. The ion exchange membranes may include, but are not limited to, commercially available membranes and membranes with chemical modifications. Non-limiting examples of such modifications are: (i) perfluorinated films with fixed pyridine or sulfonic groups; (ii) polyetherketones; (iii) polysulfonones; (iv) polyphenylene oxides; (v) polystyrene; (vi) styrene-divinyl benzene; (vii) polystyrene/acrylic based fabrics with sulfonate and quaternary ammonium cations; (viii) polyfluorinated sulfuric acid polymers; or (ix) resin-polyvinylidenedifluoride fabrics.

An anion exchange membrane as may be incorporated in an electrochemical cell can include, but is not limited to, a membrane that allows passage of anions and does not allow passage of cations. In one example embodiment, an anion exchange membrane can be a negative-valence selective membrane that allows passage of anions having a negative charge greater than a cut-off value while not allowing passage of anions having a negative charge less than the cut-off value.

Examples of anion exchange membranes as may be incorporated in an electrochemical cell can include, without limitation, those marketed under the tradename NEOSEPTA® and being of the grade AM-1, AMX, ACS and ACS-3, available from Tokuyama Corp., Tokyo, Japan; those marketed under the tradename FUMASEP® FAB, available from FuMA-Tech GmbH, Germany; and those marketed under the tradename ZIRFON®, available from Agfa Corp.

A cation exchange membrane as may be incorporated in an electrochemical cell can include a membrane that allows passage of cations and does not allow passage of anions. In one example embodiment, a cation exchange membrane can be a positive-valence selective membrane that allows passage of cations having a positive charge greater than a cut-off value while not allowing passage of cations having a positive charge less than the cut-off value.

Examples of cation exchange membranes as may be incorporated in an electrochemical cell can include, without limitation, those marketed under the tradename NEOSEPTA® and being of grade CM-1, CMX, CMS and CIMS, available from Tokuyama Corp., Tokyo, Japan; a sulfonated-tetrafluoroethylene-based fluoropolymer-copolymer commercially available under the tradename NAFION®, available from E. I. du Pont de Nemours and Company.

In one example embodiment, the electrochemical cell may be an undivided electrochemical cell, meaning that the anode and cathode are in the same electrochemical chamber. In another example embodiment, the electrochemical cell may be a divided electrochemical cell, meaning that the anode and cathode are in different electrochemical chambers separated by an ion exchange membrane.

To form the CPol derivative, the bioconjugation warhead and the halogen donor compound may undergo an electrochemical reaction in the electrochemical cell, which results in a covalent linkage between the fluorogenic compound and the halogen atom. For instance, the fluorogenic compound and the halogen donor compound may be mixed in the same electrode electrolyte solution in the electrochemical system. In one example embodiment, the fluorogenic compound and the halogen donor compound may be present in the electrode electrolyte solution at a molar ratio of from about 1:10 to about 10:1, such as from about 1:5 to about 5:1, such as from about 1:3 to about 3:1, such as from about 1:2 to about 2:1, or any range therebetween. For instance, the fluorogenic compound and the halogen donor compound may be present in the electrode electrolyte solution at a molar ratio of from about 1:5 to about 5:1. In another example embodiment, the fluorogenic compound and the halogen donor compound may be present in the electrode electrolyte solution at a molar ratio of from about 1:2 to about 2:1.

Advantageously, the oxidation potential of the halogen donor compound is lower than the oxidation potential of the fluorogenic compound. For instance, the oxidation potential of the halogen donor compound is at least about 10% lower than the oxidation potential of the fluorogenic compound, such as at least about 15% lower, such as at least about 20% lower.

In some example embodiments, the halogen atom may be selectively oxidized to form a halogen radical under mild electrochemical conditions. For instance, an electrochemically efficient voltage may be applied to the electrochemical cell to selectively oxidize the halogen donor compound without oxidizing the bioconjugation warhead. As used herein, “electrochemically efficient voltage” refers to a low voltage amount that generates a halogen radical. For instance, to selectively oxidize the halogen donor compound, an electrochemically efficient voltage of from about 0.1 V to about 2.0 V may be applied to the electrochemical cell, such as from about 0.3 V to about 1.5 V, such as from about 0.5 V to about 1.0 V. In one example embodiment, an electrochemically efficient voltage of from about 0.3 V to about 1.5 V may be applied to the electrochemical cell to selectively oxidize the halogen donor compound. Selective oxidation of the halogen donor compound forms a halogen radical.

In some example embodiments, after formation of the halogen radical, a constant voltage may continue to be applied to the electrochemical cell, which facilitates ring opening of the bioconjugation warhead. As such, the halogen radical may initiate opening the substituted CPol ring via a β-scission process, thereby forming the CPol derivative.

In some example embodiments, the resulting CPol derivative may act as an electrophilic warhead. For instance, the electrophilic warhead may include a β-substituted ketone. In one example embodiment, the electrophilic warhead have the following Formula I-#:

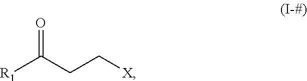

wherein R₁ may be selected from a group consisting of hydroxyl, amino, alkoxy, carboxy, benzyl, phenyl, nitro, thiol, thioalkoxy, halogen, alkyl, aryl, alkenyl, alkynyl, halogen, C₁₋₆alkyl, phenyl, perdeuterated phenyl, benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl, 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbanoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, or a combination thereof; and
wherein X may be a halogen.

In one example embodiment, the electrophilic warhead may include, but is not limited to, 1-(6-methoxynaphthalen-2-yl)cyclopropan-1-ol ketone (Npt-ketone), 7-hydroxy-3-(1-hydroxycyclopropyl)-2H-chromen-2-one ketone (Coumarin-ketone), 3-iodo-1-phenylpropan-1-one, 3-iodo-1-(p-tolyl)propan-1-one, 4-iodo-1-phenylbutan-2-one, 1-iodooctan-3-one, 1-iodopentan-3-one, 3-iodo-1-(4-methoxyphenyl)propan-1-one, 3-iodo-1-(4-(trifluoromethyl)phenyl)propan-1-one, 3-iodo-1-(3-methoxyphenyl)propan-1-one, 3-iodo-1-(3-(trifluoromethyl)phenyl)propan-1-one, 1-iodo-4-methylpentan-3-one, N-(2-hydroxyethyl)-6-iodo-N, N-dimethyl-4-oxohexan-1-aminium bromide, or a combination thereof. In another example embodiment, the electrophilic warhead may include, but is not limited to, 3-bromo-1-phenylpropan-1-one, 3-bromo-1-(p-tolyl)propan-1-one, 4-bromo-1-phenylbutan-2-one, 1-bromooctan-3-one, 1-bromopentan-3-one, 3-bromo-1-(4-methoxyphenyl)propan-1-one, 3-bromo-1-(4-(trifluoromethyl)phenyl)propan-1-one, 3-bromo-1-(3-methoxyphenyl)propan-1-one, 3-bromo-1-(3-(trifluoromethyl)phenyl)propan-1-one, 1-bromo-4-methylpentan-3-one, N-(2-hydroxyethyl)-6-bromo-N, N-dimethyl-4-oxohexan-1-aminium bromide, or a combination thereof.

In one example embodiment, the electrophilic warhead may be 1-(6-methoxynaphthalen-2-yl)cyclopropan-1-ol ketone (Npt-ketone). For instance, Npt-Ketone may have the following Formula (I-1a):

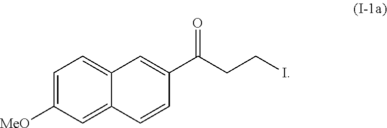 [SMILES: COc1ccc2cc(C(=O)CCI)ccc2c1]

In one example embodiment, the electrophilic warhead may be, 7-hydroxy-3-(1-hydroxycyclopropyl)-2H-chromen-2-one ketone (Coumarin-ketone). For instance, coumarin-ketone may have the following Formula (I-2a):

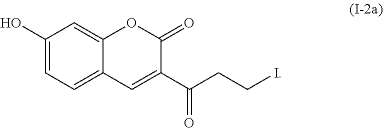 [SMILES: O=C(CCI)c1cc2ccc(O)cc2oc1=O]

In some example embodiments, the electrophilic warhead may be selectively conjugated to a biomolecule via an electrochemical reaction. For instance, the electrophilic warhead may be conjugated to the N-terminus of a biomolecule. In another example embodiment, the electrophilic warhead may be conjugated to the C-terminus of a biomolecule. For instance, the electrophilic warhead may be conjugated to a biomolecule as a biorthogonal tag. In one example embodiment, the electrophilic warhead disclosed herein may be conjugated resulting in a conjugated biomolecule. To conjugate the electrophilic warhead to a biomolecule, an electrochemically efficient voltage of from about 0.1 V to about 2.0 V may be applied to the electrochemical cell, such as from about 0.3 V to about 1.5 V, such as from about 0.5 V to about 1.0 V, or any range therebetween.

The electrophilic warhead disclosed herein may be conjugated to any compatible biomolecule to be labeled. In some example embodiments, the biomolecule may have an average molecular weight (Mw) of at least about 1,000 g/mol, such as at least about 2,000 g/mol, such as at least about 10,000 g/mol, such as at least about 20,000 g/mol, such as at least about 50,000 g/mol, such as at least about 100,000 g/mol, such as at least about 250,000 g/mol, such as at least about 500,000 g/mol. In one example embodiment, the biomolecule may have an average Mw in the range of about 1,000 g/mol to about 30,000 g/mol, or any range therebetween.

The fluorogenic compound disclosed herein may be a valuable tool for real-time cell imaging or various other bioconjugation applications. Advantageously, the conjugated fluorogenic compound may be utilized in visualizing a sample. For instance, the sample may be a tissue sample, bodily fluid, cell, cell culture, microvesicle, or any combination thereof. In one example embodiment, the sample may be a tissue sample. In another example embodiment, the sample may be a cell. In yet another example embodiment, the sample may be a cell culture. For instance, the conjugated fluorogenic compound may be incorporated into mammalian cells via the biosynthetic process. Also, referred to herein as metabolic incorporation of the fluorogenic compound into the cells.

In one example embodiment, the fluorogenic compound disclosed herein may be useful for imaging live cells in real time. In one example embodiment, the fluorogenic compound may be subjected to an oxidative condition. The oxidation condition may include, but is not limited to, electrochemical oxidation, iodine oxidation, N-Iodosuccinimide (NIS) oxidation, or a combination thereof. For instance, the fluorogenic compound disclosed herein may be incorporated into a cell via incubating the live cells with the fluorogenic compound for a sufficient time to allow for metabolic incorporation of the fluorogenic compound. As such, incubation of the live cells with the fluorogenic compound may electrochemically promote the formation of the conjugated biomolecule within the live cells. To image the live cells, the cell may be electrochemically reacted with the conjugated fluorogenic compound and a dye. For instance, the dye may be a fluorescent dye. In one example embodiment, for instance, the fluorescent dye may be fluorescein thiosemicarbazide.

In one example embodiment, imaging the fluorogenic compound may include measuring the fluorescence intensity change of the fluorogenic compound using methods understood in the art.

In one example embodiment, the temperature incubating the live cells with the fluorogenic compound may be from about 20° C. to about 40° C., such as from about 25° C. to about 35° C., or any range therebetween. In one example embodiment, for instance, the temperature incubating the live cells with the fluorogenic compound may be 30° C. In another example embodiment, for instance, the temperature incubating the live cells with the fluorogenic compound may be 37° C.

In one example embodiment, the sufficient time to allow for metabolic incorporation of the fluorogenic compound via incubation may be from about 30 minutes to about 24 hours, such as from about 1 hour to about 20 hours, such as from about 3 hours to about 15 hours, or any range therebetween. For instance, in one example embodiment, the incubating time may be from about 1 hour to about 20 hours. In another example embodiment, the incubating time may be from about 3 hours to about 15 hours.

Beneficially, disclosed herein is the development of controllable fluorogenic compounds based on cyclopropanol (CPol) scaffolds and their application in bioconjugation reactions. Specifically, the compositions disclosed herein may be oxidant- or electrochemistry-activatable CPol-based fluorophores that undergo ring-opening under oxidative or electrochemical conditions to generate electrophilic β-halide-substituted ketones, which in turn enable selective conjugation with nucleophilic biomolecules such as peptides and proteins. This platform facilitates spatiotemporally controlled labeling of biomolecules in vitro and live-cell environments.

The preceding description is exemplary in nature and is not intended to limit the scope, applicability or configuration of the disclosure in any way. Various changes to the described embodiments may be made in the function and arrangement of the elements described herein without departing from the scope of the disclosure.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

The present disclosure may be better understood with reference to the following examples.

### EXAMPLES

### Materials and Methods

### MALDI-TOF MS Analysis

Mass spectra were generated with a MALDI-TOF mass spectrometer (rapifleX MALDI Tissuetyper from BRUKER) equipped with a solid-state smart beam and operated in a linear delayed extraction positive ion mode. The samples of conjugated peptide were concentrated using IMCStips (1 mL, IMCS company) and eluted with 70% acetonitrile/0.1% trifluoracetic acid (TFA) following the manufacturer protocol. Of note, the samples of conjugated proteins and viruses were denatured by incubating with guanidinium chloride (8 M) for 5 min at RT, followed by the removal of salts and elution using IMCStips. The resultant samples were spotted on a MALDI plate. These spots were analyzed by MALDI-TOF MS spectrometer with the matrix (Vₛₐₘₚₗₑ:Vₘₐₜᵣᵢₓ=1:1). Matrix of sinapic acid in 50% acetonitrile/0.1% TFA was used for protein measurements, and the matrix of α-Cyano-4-hydroxycinnamic acid in 50% acetonitrile/0.1% TFA was used for peptide measurements.

### Electrochemical-Promoted Ring-Opening Reactions of CPol Derivatives

In a divided H-cell equipped with a stir bar, CPol derivatives (0.2 mmol) and NaI (0.36 mmol) or NaBr (0.36 mmol) were mixed in 6 mL acetonitrile/water (1:1) solvents containing 0.1 M Na₂SO₄ electrolyte. The H-cell was equipped with a carbon sheet as an anode, Pt sheet as the cathode, and Ag/AgCl as a reference electrode. Then, the H-cell was exposed to a nitrogen atmosphere for 10 min. The reaction mixture was stirred and electrolyzed at a constant potential (0.8 V for NaI, 1.2 V for NaBr) for 8 h at room temperature. After stopping the reaction, the reaction mixture was extracted with EtOAc (3×10 mL) and saturated NH₄Cl (1×15 mL). The organic layer was dried over Na₂SO₄ and concentrated in a vacuum.

Stain-Free in-Gel Fluorescence SDS-PAGE Analysis Using Fluorogenic Conjugates

For electrochemical conjugation of CPol derivatives with proteins, biomolecules (50 μM) in 0.1 M phosphate-buffered saline (pH 7.0) were mixed with CPol derivatives and various anions under electrochemical conditions. The mixture was treated under constant potential at 1.2 V for 3 hours at room temperature. Meanwhile. After the reaction, the mixture was incubated at 4° C. overnight. Next, the mixture was concentrated using Amicon® Ultra Centrifugal Filters (MWCO 3 kDa) at 4° C. (7,500 rpm, 30 min). The cut-off conjugated proteins (20 μg) were mixed with Laemmli SDS-sample buffer (4×, Reducing, Boston Bioproducts) and resolved by 10% SDS-PAGE. For the detection of fluorogenic conjugates, fluorescent signals were acquired using the ChemiDoc MP Imaging System (Bio-Rad).

### Example 1

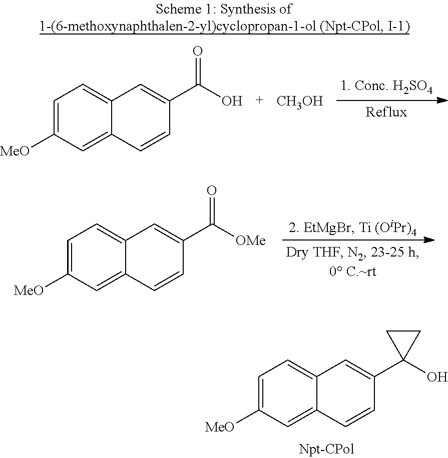

Step 1: To a solution of 2-naphthoic acid (25.83 g, 150.00 mmol) in methanol (150 mL), concentrated sulfuric acid (7.5 mL) was added. The reaction mixture was then heated to reflux at 80° C. for 16 hours. After completion, the solvent was removed under reduced pressure, and the resulting residue was treated with water (200 mL). Sodium bicarbonate was added until the pH of the mixture reached 9. The aqueous phase was extracted four times with dichloromethane, and the combined organic layers were dried over magnesium sulfate. Finally, the solvent was removed under reduced pressure to yield methyl-2-naphthoate.

Step 2: Ethylmagnesium bromide (10.6 mmol, 10.6 mL, 1 M in THF) was added dropwise over 1 h at 4° C. to a solution of ester (5 mmol) and titanium tetraisopropoxide (1 mmol, 0.3 mL) in THF (15 mL) under N₂ protection. The mixture was then stirred at room temperature for 23-25 h. Then the reaction was quenched by the addition of water (about 15 mL) under an ice bath while stirring. After that, the mixture was filtered by vacuum filtration, obtaining a liquid solution. The solution was then extracted with anhydrous diethyl ether (3×20 mL) and saturated NH₄Cl (1×40 mL). The organic layers were combined and dried with Na₂SO₄, and the crude mixture was purified by column chromatography on silica gel (Hexane:EtOAc=20:1) to yield the Npt-CPol I-1 in isolation yield of 51%.

### Example 2

### Scheme 2: Synthesis of 7-hydroxy-3-(I-hydroxycyclopropyl)-2H-chromen-2-one (Coumarin-CPol, I-2)

Step 1: To a solution of 2,4-dihydroxy benzaldehyde (15.0 g, 0.108 mol, 1.0 eq) in ethanol (50 mL) was added ethyl acetoacetate (13.62 mL, 0.108 mol, 1.0 eq), followed by a catalytic amount of piperidine (0.1 mL). The resulting mixture was refluxed for 18 h. The completion of the reaction was checked by thin-layer chromatography (TLC). After completion of the reaction, the reaction mixture was allowed to cool down to room temperature and concentrated on a rotavapor. The viscous liquid obtained was poured into ice-cold water to give a solid. The solid obtained was filtered, washed with water, dried, and recrystallized from ethanol to give compound 3-acetyl-7-hydroxy-2H-chromen-2-one as a green solid in a yield of 45%.

Step 2: To the stirred solution of 3-acetyl-7-hydroxy-2H-chromen-2-one (1 mmol) in DMF (5 mL) was added imidazole (2 mmol) at room temperature, and after 5 minutes, TBSOTf (1.2 mmol) was added, and stirring was continued until completion of the reaction (TLC monitoring). Next, water (30 mL) was added to the reaction mass and extracted with diethyl ether (2×30 mL). The combined organic layer was washed with brine (50 mL), dried (Na₂SO₄), and concentrated under vacuum, giving respective compound quantitative yields.

Step 3: TMSOTf (1.2 mmol) was added to a solution of ketone (1 mmol) and Et₃N (1.2 mmol) in DCM (2 mL) at 0° C. The cooling bath was removed, and the solution was stirred for 40 min at room temperature. After being diluted with DCM, the reaction mixture was poured into a saturated aqueous NaCl solution (15 mL) and extracted with DCM (3×20 mL). The combined organic extracts were dried over anhydrous MgSO₄, filtered, and concentrated under reduced pressure, giving respective compound quantitative yields.

### Example 3

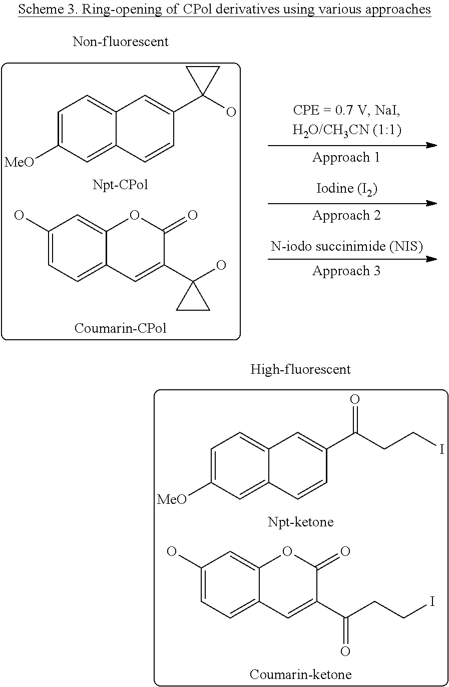

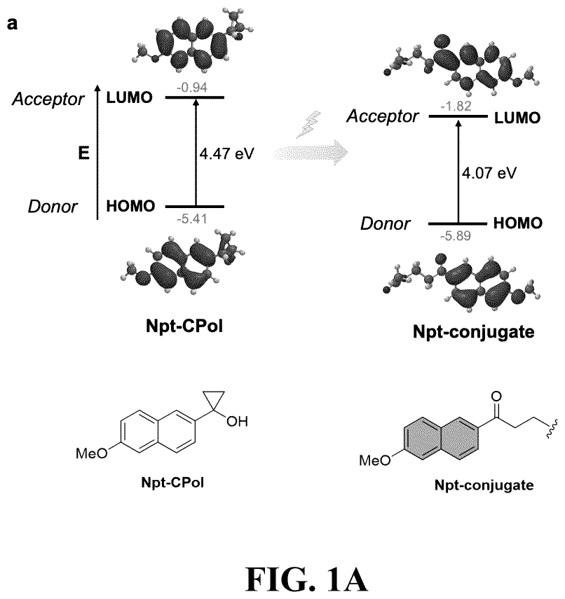

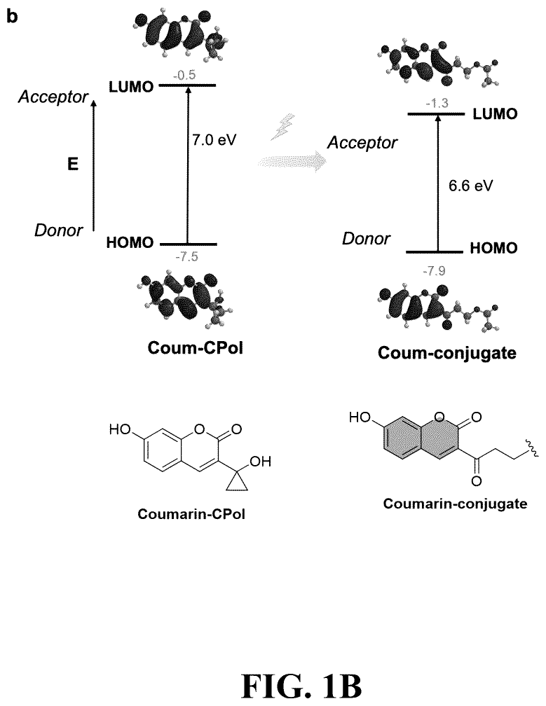

Npt-CPol I-1 and Coumarin-CPol I-2 exhibit very weak fluorescence due to the absence of intramolecular charge transfer (ICT)². To enhance the fluorescence of these CPol derivatives, the CPol ring can be opened to generate a ketone, which facilitates ICT through its electron-withdrawing effect, as predicted by Spartan calculations (FIGS. 1A and 1B). Interestingly, the CPol ring can be opened through various approaches—such as electrochemical stimulation, iodine oxidation, or NIS oxidation—to generate β-substituted ketones, thereby rendering I-1 and I-2 potential fluorogenic dyes. (Scheme 3).

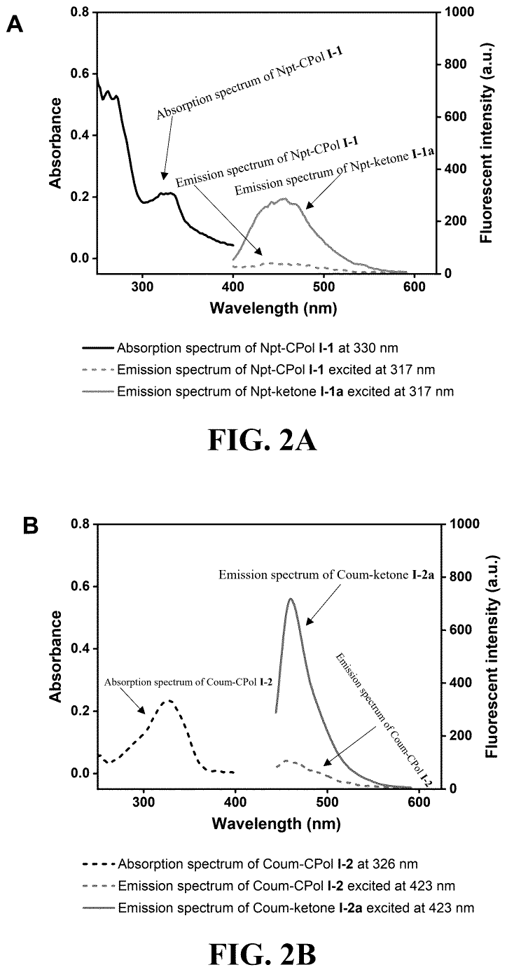

The fluorescence intensities of I-1 and I-2, as well as their ring-opened products I-1a and I-2a, were measured using a fluorescence spectrometer. The results showed a significant increase in fluorescence intensity following the ring-opening reactions of I-1 (FIG. 2A) and I-2 (FIG. 2B), consistent with the predictions from Spartan calculations.

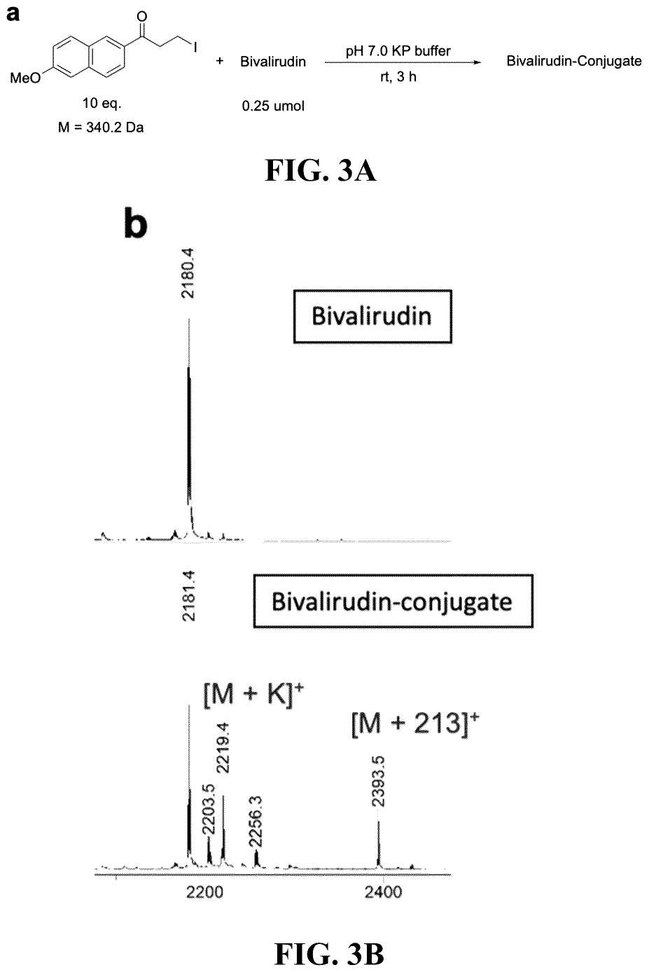

Consequently, the substitution reaction of this halide-containing ketone with biomolecules was investigated to assess the potential of this fluorogenic transformation for bioconjugation applications (FIG. 3A). It was shown that the successful conjugation of Npt-ketone I-1a to bivalirudin was confirmed by MALDI analysis (FIG. 3B).

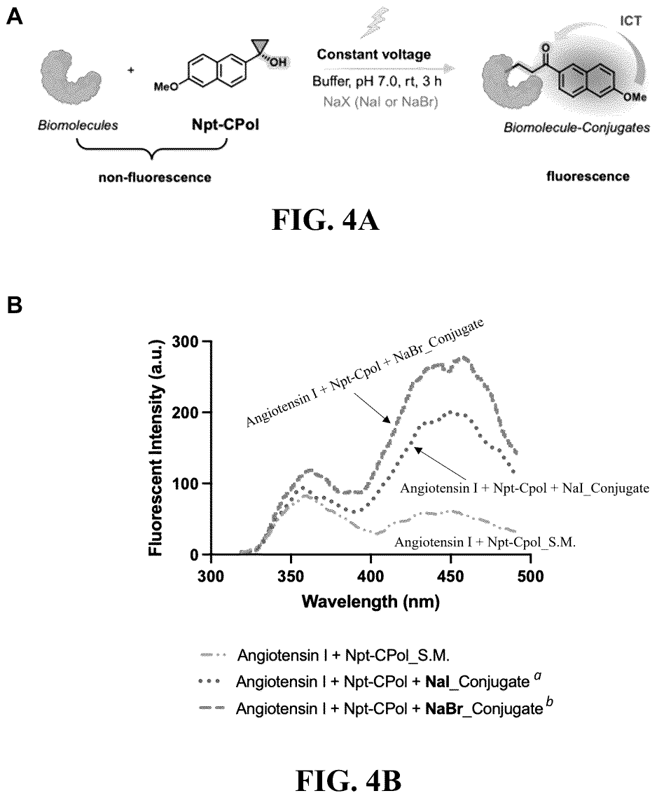

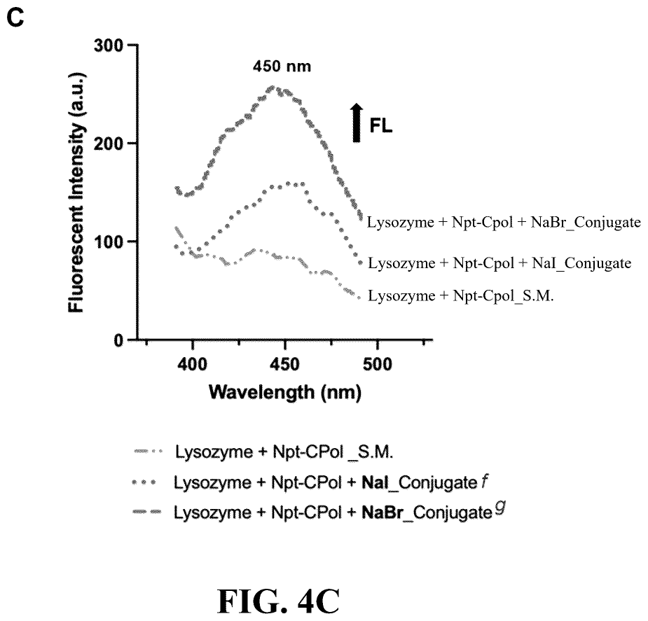

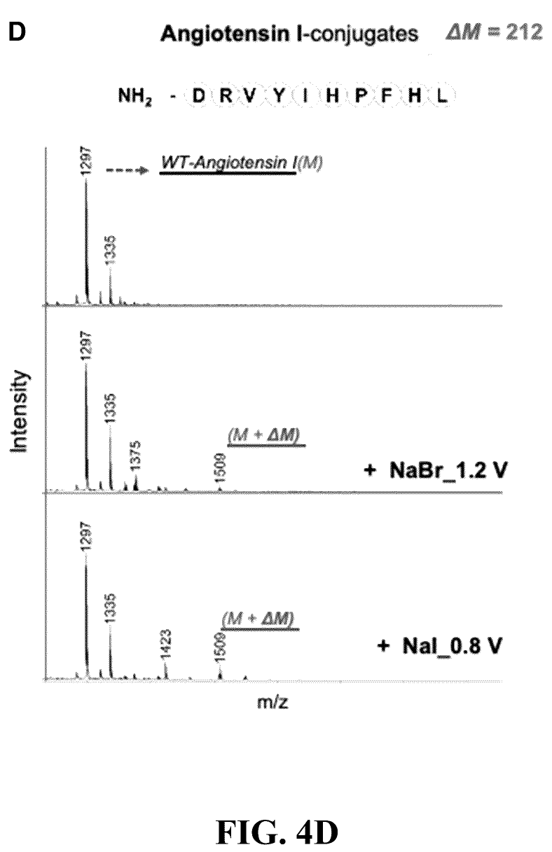

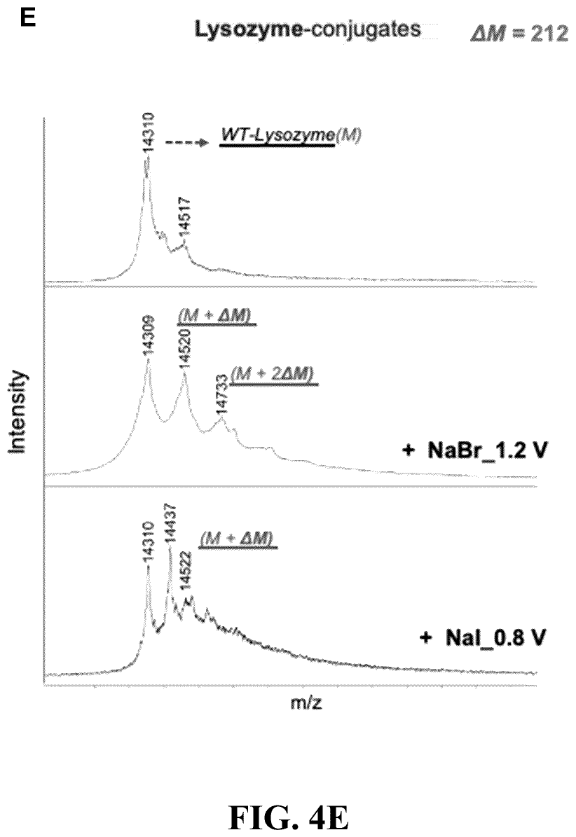

Based on the studies of the ICT occurrence in the ring-opening of I-1 and I-2 and the bioconjugation reaction between Npt-ketone I-1a and bivalirudin, it was reasoned that fluorogenic bioconjugation between Npt-CPol I-1 and biomolecules could be achieved under ring-opening conditions. Here, we employed electrochemistry as a mild and in situ method to induce ring opening of the CPols (FIG. 4A). Therefore, the fluorogenic bioconjugation reaction between Npt-CPol and the peptide angiotensin I was investigated, as well as the protein lysozyme, under electrochemical conditions using fluorescent spectrometer. The results showed an increase in fluorescence for the biomolecule-conjugated Npt, with a wavelength shift to 450 nm (FIGS. 4B and 4C). (Fluorescent spectroscopy conditions: excitation wavelength is 317 nm, the voltage is 800 V). Additionally, the bioconjugates were confirmed by MALDI measurement (FIGS. 4D and 4E). (Electrochemical conditions: a Angiotensin I (50 μM), fluorogenic dyes (20 eq of I-1) and NaI (20 eq) at a constant voltage of 0.8 V; b The fluorogenic bioconjugation was performed between angiotensin I (50 μM), fluorogenic dyes (20 eq of I-1) and NaBr (50 eq) at a constant voltage of 1.2 V; f The fluorogenic bioconjugation was performed between lysozyme (50 μM), fluorogenic dyes (20 eq of I-1) and NaI (20 eq) at a constant voltage of 0.8 V; g The fluorogenic bioconjugation was performed between lysozyme (50 μM), fluorogenic dyes (50 eq of I-1) and NaBr (50 eq) at constant voltage of 1.2 V.)

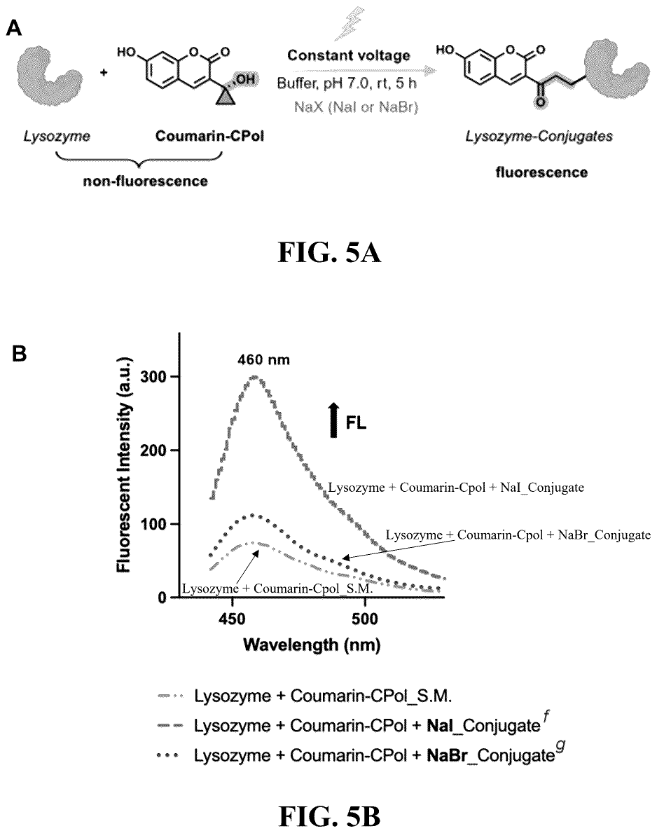

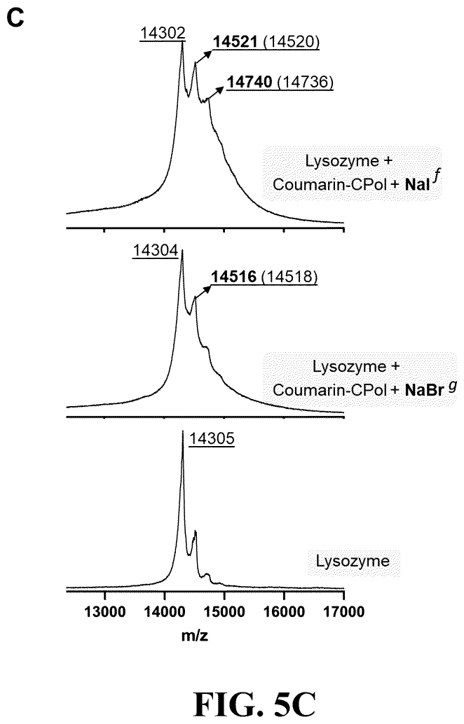

In addition to Npt-CPol, many other fluorophores containing the CPol ring structure can be synthesized. For example, coumarin is a popular profluorophore due to its small size, biocompatibility, and ease of synthetic manipulation. The fluorogenic reaction between coumarin-CPol I-2 and the protein lysozyme has been investigated (FIG. 5A). Results demonstrated an increase in fluorescence when treating I-2 with electrochemistry and lysozyme (FIG. 5B). (Fluorescent spectroscopy conditions: excitation wavelength is 423 nm, the voltage is 800 V). The lysozyme-coumarin conjugates were confirmed by MALDI analysis (FIG. 5C). (Electrochemical conditions: f The fluorogenic bioconjugation was performed between lysozyme (50 μM), fluorogenic dyes (20 eq of 1-2) and NaI (20 eq) at a constant voltage of 0.8 V; g The fluorogenic bioconjugation was performed between lysozyme (50 μM), fluorogenic dyes (50 eq of 1-2) and NaBr (50 eq) at constant voltage of 1.2 V.)

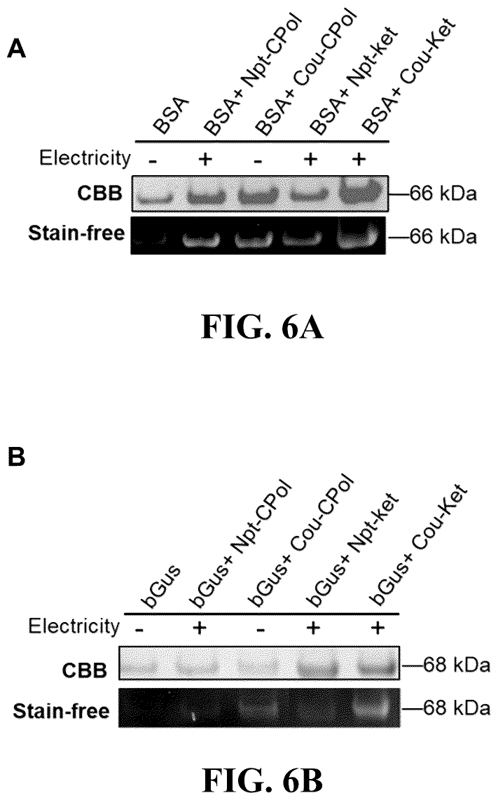

With BSA (FIG. 6A) and bGus (FIG. 6B) as model proteins, stain-free in-gel fluorescence analysis confirmed successful fluorogenic bioconjugation of I-1 and 1-2 under electrochemical conditions, demonstrating the utility of these probes for direct biomolecular labeling and monitoring.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A fluorogenic compound, the fluorogenic compound comprises Formula (1): 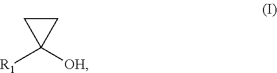
wherein R₁ is selected from a group consisting of 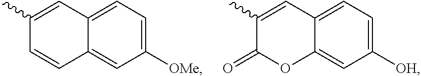
hydroxyl, amino, alkoxy, carboxy, benzyl, phenyl, nitro, thiol, thioalkoxy, halogen, alkyl, aryl, alkenyl, alkynyl, halogen C₁₋₆alkyl, phenyl, perdeuterated phenyl benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl, 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbamoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, or a combination thereof. In one example embodiment, R1 may include, but is not limited to, phenyl, toyl, benzyl, phenyl, methoxyphenyl, methyl, fluoromethyl, trifluoromethyl, propyl, isopropyl, or a combination thereof.

2. The fluorogenic compound of claim 1, wherein R₁ is

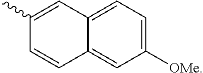

3. The fluorogenic compound of claim 1, wherein R₁ is

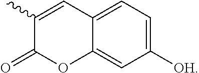

4. The fluorogenic compound of claim 1, wherein the fluorogenic compound of Formula (I) is

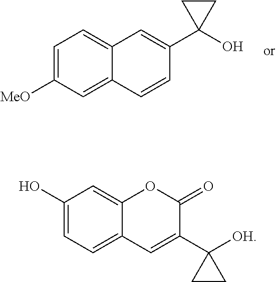 [SMILES: COc1ccc2cc(C3(O)CC3)ccc2c1 ; O=c1oc2cc(O)ccc2cc1C1(O)CC1]

5. The fluorogenic compound of claim 1, wherein the fluorogenic compound of Formula (I) is

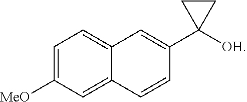 [SMILES: COc1ccc2cc(C3(O)CC3)ccc2c1]

6. The fluorogenic compound of claim 1, wherein the fluorogenic compound of Formula (I) is

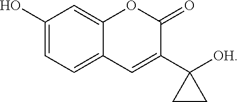 [SMILES: O=c1oc2cc(O)ccc2cc1C1(O)CC1]

7. A method of visualizing a biomolecule, the method comprising:
conjugating a biomolecule with a sample comprising a fluorogenic compound having the structure of Formula (1): 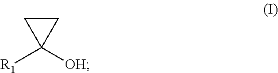
incubating the biomolecule with the fluorogenic compound for a sufficient time to allow for fluorogenic bioconjugation of the fluorogenic compound;
subjecting the fluorogenic compound to an oxidative condition;
contacting the fluorogenic compound with a dye; and
imaging the fluorogenic compound and the dye, thereby determining fluorescence intensity change of the fluorogenic compound.

8. The method of claim 7, wherein R₁ is selected from a group consisting of 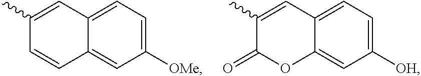
hydroxyl, amino, alkoxy, carboxy, benzyl, phenyl, nitro, thiol, thioalkoxy, halogen, alkyl, aryl, alkenyl, alkynyl, halogen C₁₋₆alkyl, phenyl, perdeuterated phenyl benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbamoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, or a combination thereof. In one example embodiment, R1 may include, but is not limited to, phenyl, toyl, benzyl, phenyl, methoxyphenyl, methyl, fluoromethyl, trifluoromethyl, propyl, isopropyl, or a combination thereof.

9. The method of claim 7, wherein R₁ is

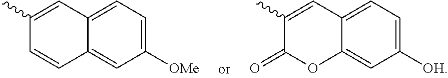

10. The method of claim 7, wherein the fluorogenic compound of Formula (I) is

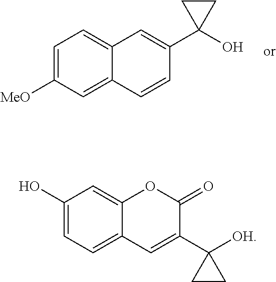 [SMILES: COc1ccc2cc(C3(O)CC3)ccc2c1 ; O=c1oc2cc(O)ccc2cc1C1(O)CC1]

11. The method of claim 7, wherein the fluorogenic compound of Formula (I) is

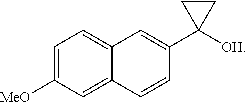 [SMILES: COc1ccc2cc(C3(O)CC3)ccc2c1]

12. The method of claim 7, wherein the fluorogenic compound of Formula (I) is

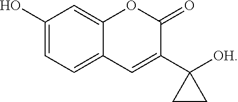 [SMILES: O=c1oc2cc(O)ccc2cc1C1(O)CC1]

13. The method of claim 7, wherein the sample comprises a tissue sample, bodily fluid, cell, cell culture, microvesicle, or any combination thereof.

14. The method of claim 7, wherein the sample comprises a cell.

15. The method of claim 7, wherein the sample comprises a cell culture.

16. The method of claim 7, wherein the biomolecule comprises nucleic acids, proteins, peptides, or viruses.

17. The method of claim 7, wherein the dye comprises a fluorescent dye.

18. The method of claim 7, the fluorogenic compound further comprising a halogen donor compound.

19. The method of claim 18, wherein the halogen donor compound comprises sodium iodide, sodium bromide, lithium bromide, lithium iodide, potassium bromide, potassium iodide, tetraethylammonium bromide, tetraethylammonium chloride, or a combination thereof.

20. The method of claim 7, wherein the oxidative condition comprises electrochemical oxidation, iodine oxidation, N-Iodosuccinimide (NIS) oxidation, or a combination thereof.
